(19) United States
(12) Plant Patent Application Publication
(10) Pub. No.: US 2009/0320169 P1
(43) Pub. Date: Dec. 24, 2009

(54) Labiatae plant named 'Guizhou Qizhen Cao'

(50) ;

(76) Inventors: Linxiang Mou, Anshun City (CN); Xiuying Lin, Anshun City (CN)

Correspondence Address: DAVID AND RAYMOND PATENT FIRM, 108 N. YNEZ AVE., SUITE 128, MONTEREY PARK, CA, 91754, US

(21) Appl. No.: 12/215,017

(22) Filed: Jun. 23, 2008

(51) Int. Cl. A01H 5/00

(52) U.S. Cl. PLT/258

(57) ABSTRACT

‘Guizhou Qizhen Cao’ is a distinctive variety of Labiatae Plant, which is characterized by its appearance and color of the whole plant.

### LATIN NAME

Labiatae Plant

### VARIETY DENOMINATION

Guizhou Qizhen Cao

### BACKGROUND OF THE INVENTION

The present invention relates to a new and distinct variety of herbal Labiatae plant, which is a grass-like plant given the varietal denomination of ‘Guizhou Qizhen Cao’.

The new herb Guizhou Qizhen grass discovered by the applicant differs from related herbal plants including Sallvia Multiorrhiza, Bge (Dan Shen), Scutellaria Indica L. (Han Sin Cao), Scutellaria Barbata Don (Ban Zhi Lian), and Scutellaria Discolor Colebr (Wa Er Cao), in which the Dan Shen has pulp roots and white flowers, the Wa Er Cao has a soft hairy stem, fibrous roots and blue flowers, and the Ban Zhi Lian is arranged like a toothbrush that has fibrous roots and blue flowers

### SUMMARY OF THE NEW VARIETY

‘Guizhou Qizhen Cao’ is a distinctive variety of Labiatae plant, which is characterized by its quadri-angular stem, fibrous roots with beaded root nodules grown thereon, purplish red peduncles, and purplish red flowers

### BRIEF DESCRIPTION OF THE DRAWINGS

FIG. 1 is a color photograph of the new invention, ‘Guizhou Qizhen Cao’, showing the whole plant in the field.

FIG. 2 is a color photograph of the new invention, ‘Guizhou Qizhen Cao’, showing the whole plant.

FIG. 3 is a color photograph of the invention showing the whole plant with roots.

FIG. 4 is a color photograph of the invention showing its upright extending stem and flowers.

FIG. 5 is a color photograph of the invention showing the appearance of its flowers in purplish red color.

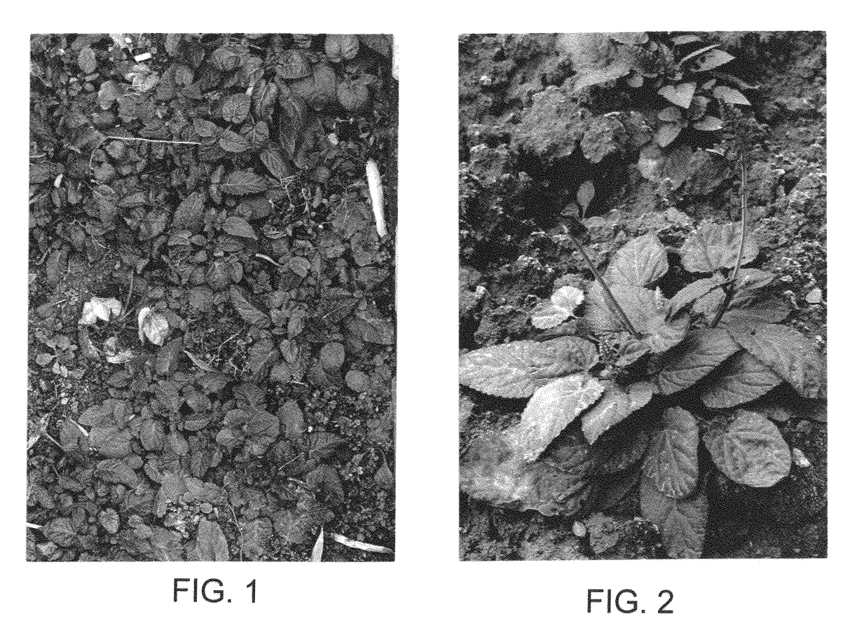

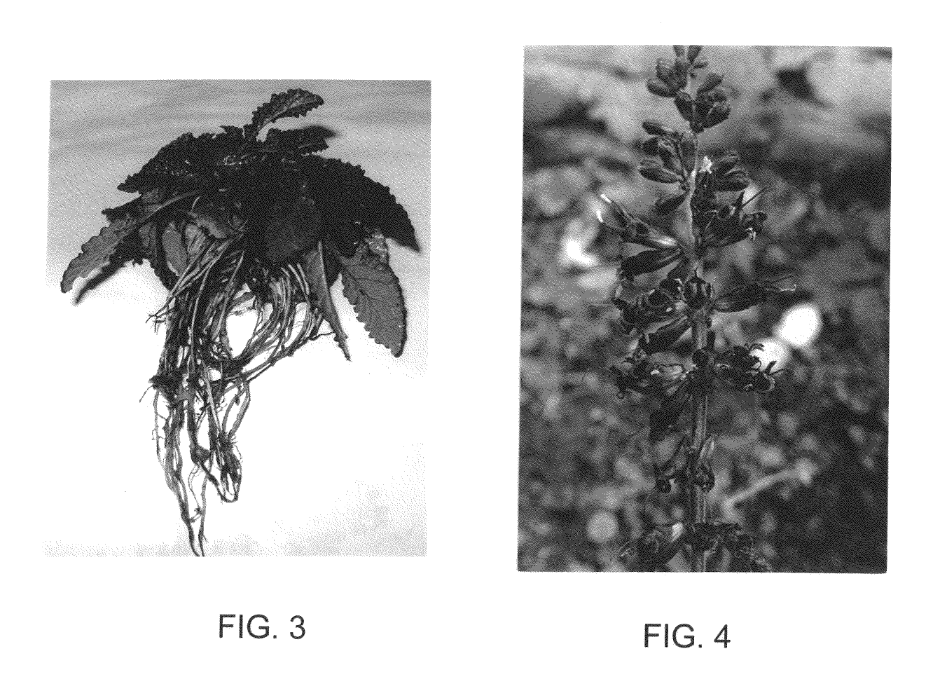

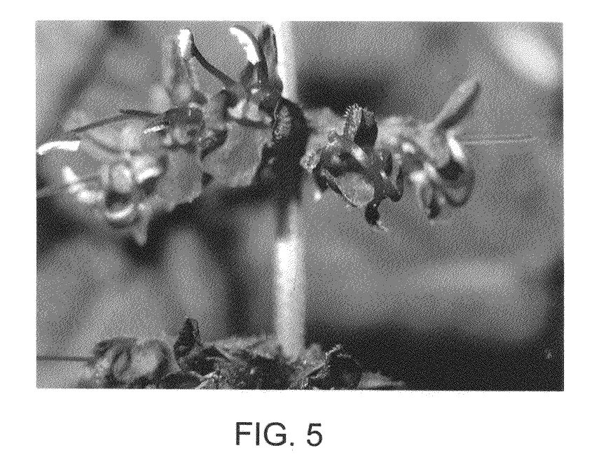

### DETAILED DESCRIPTION

The accompanying colored photographs illustrate the overall appearance of the new and distinct variety of the herbal Labiatae Plant, which is a grass-like plant, known as ‘Guizhou Qizhen Cao’. The descriptions disclosed herein are based upon observations of the plant growth.

The size of the whole plant has a height of 10-30 cm. The stem which has a quadric-angular shape is an erect stem or a stolon stem. The roots are fibrous roots with beaded root nodules grown thereon. The leaves are in oval shape and grown from the roots. Each leaf has a heart shaped leaf base and a peripheral edge with shallow round teeth. Each plant has single leaf or 1-2 pairs of compound leaf. Each leaf has a leafstalk having a length of 8-12 cm. The two blade surfaces of each leaf are hairless and have plenty of wrinkles. The color of the leaves is green or purplish red. The plant has about 30 leaves at the stem portion while the leaves at the peduncle are in pair manner. The flowers are arranged in circular taper manner and there are 15-22 rounds of flowers, wherein each round has 6 flowers and each flower has a size of 8×3 mm. The flowers are lip style flowers, each having a tubular shape and two slits at the lower lip. Each flower has two stamens. The style of each flower is grown from the ovary. The flowers are arranged at the top portion of the stem to form a whole series of flowers. The corolla of each flower is in purplish red color. The nuts are small and each has an elliptical shape and a dark red color. The nut is matured within the calyx. The blossom period is 6-7 months and the period of bearing fruit is 8-9 months.

The plant grows from seedling propagation and independently. The plant can be cultured and propagated in garden. The whole plant can be used as oriental medicine. The growing condition prefers windy, warm and wet environment. The rest period of the plant is winter that the plant stops to grow and to grow leaf temporally. The plant has a slight fragrant and a bitter taste. Each acre may grow more than 300-400 kg of fresh plants for two seasons per year. The plant is preferred to start planting in March of each year, in which it needs 40 days for budding and takes about 6 months from bud to mature for picking. The plant begins to grow peduncle and blossom in May-June each year and the seeds get mature between August and September.

These features and other characteristics of the plant are apparent from the figures.

What is claimed is:

1. A new and distinct variety of Labiatae Plant named ‘Guizhou Qizhen Cao’, substantially as described and illustrated.
